(19) United States
(12) Patent Application Publication
(10) Pub. No.: US 2022/0361735 A1
(43) Pub. Date: Nov. 17, 2022

(54) CLIP DEVICE FOR ENDOSCOPE

(71) Applicant: OLYMPUS MEDICAL SYSTEMS CORP., Tokyo (JP)

(72) Inventors: Shogo SHINDO, Tokyo (JP); Tatsunori TSUNETO, Tokyo (JP); Shinya ANSAI, Tokyo (JP); Yoshitsugu UEKUSA, Tokyo (JP)

(73) Assignee: OLYMPUS MEDICAL SYSTEMS CORP., Tokyo (JP)

(21) Appl. No.: 17/677,219

(22) Filed: Feb. 22, 2022

Related U.S. Application Data
(60) Provisional application No. 63/187,651, filed on May 12, 2021.

(51) Int. Cl. A61B 1/00; A61B 1/018

(52) U.S. Cl. CPC A61B 1/00087; A61B 1/018

(57) ABSTRACT

Treatment device with clip is reciprocally closed and opened in one operation and locked in a second, distinct operation. The clip has a first arm and second arm which are configured to move in a direction toward and away from each other to close and open the clip; a wire connected to a proximal end of the clip configured to reciprocally move within a sheath to close and open the clip; and one or more latch elements on the clip configured to lock the clip in a closed state. Operating the wire does not lock the clip in a closed state in a single operation. Rather, the clip can be locked by another operation that is distinct from operating the wire for reciprocal movement, such as operating a stopper, an inner sheath reciprocally movable to slid over the clip arms, or a locking ring slidable over the clip arms.

### CROSS-REFERENCE TO RELATED APPLICATION

This application claims the benefit of U.S. Provisional Application No. 63/187,651 filed May 12, 2021, the entire disclosure of which is hereby incorporated by reference herein.

### TECHNICAL FIELD

The present disclosure relates to a clip device for gripping tissue of a patient that includes a clip configured with latches to lock the clip in the closed state for use with surgical tools such as endoscopes.

### BACKGROUND

In the discussion that follows, reference is made to certain structures and/or methods. However, the following references should not be construed as an admission that these structures and/or methods constitute prior art. Applicant expressly reserves the right to demonstrate that such structures and/or methods do not qualify as prior art against the present invention.

Various treatment tools can be used with surgical tools, such as endoscopes, for a variety of therapeutic procedures such as to prevent tissue bleeding, closing perforations, and other surgical procedures. Such treatment tools can grasp tissue within a clip and lock the clip by operation of a wire connected to the clip. However, users of such tools can unintentionally lock the clip when operating the wire during a therapeutic procedure.

Hence, a continuing need exists for treatment tools that can be used with endoscopes which can minimize locking of clips when not intended.

### SUMMARY OF THE DISCLOSURE

An advantage of the present disclosure includes a treatment tool or device that includes a clip than can be reciprocally closed and opened in one operation and locked in a separate operation, distinct from the operation of reciprocally closing and opening the clip.

These and other advantages are satisfied, at least in part, by a clip device comprising a clip having a first arm and second arm in which the first arm and second arm are configured to move in a reciprocal direction toward and away from each other to close and open the clip; a wire connected to a proximal end of the clip configured to reciprocally move within a sheath to close and open the clip; and one or more latch elements on the clip configured to lock the clip in a closed state. Advantageously, operating the wire does not lock the clip in a closed state in a single operation. Rather, the clip can be locked by another operation, e.g., a second operation, such as by operating a stopper or by operating a control member reciprocally movable within the sheath to slid over the clip arms or to slid a locking ring over the clip.

Another aspect of the present disclosure includes a method of operating a clip device that includes closing a clip having a first arm and second arm biased in an open state and having one or more latch elements by pulling a wire connected to a proximal end of the clip to retract the clip in a tube or pipe (tube and pipe are used interchangeably herein); and locking the clip in a closed state by moving a stopper or by an operation other than pulling the wire to close the clip. The method can further include locking the clip by sliding a locking ring distally over the one or more latch elements and/or locking the clip by sliding a control member, e.g., an inner sheath, over the clip to lock mating latch elements.

Additional embodiments of the present disclosure include one or more of the following features individually or combined. For example, the clip device of claim can further include a locking ring that can slidably move over the one or more latch elements to lock the clip. The locking ring and the one or more latch elements can have complementary mating portions, which lock the locking ring to the one or more latch elements to lock the clip. In some embodiments, the first arm or second arm or both arms can include a projection that prevent(s) the arms from fully retracting in a tube distally connected to the sheath. In other embodiments, the clip device can further include a control member, e.g., an inner sheath, reciprocally movable within the sheath by operation of a control knob that is configured to slidably move the locking ring over the one or more latch elements to lock the clip. In still further embodiments, the first arm includes a first latch element and the second arm includes a second latch element opposite the first latch element and configured to engage the first latch element to lock the clip. In other embodiments, the clip device can further include a tube connected to a distal end of the sheath, which does not disconnect from the sheath.

In further embodiments, the clip device can further include a stopper connected to the wire, such as to a distal end region of the wire and the device can further include a control member, e.g., an inner sheath, reciprocally movable within the sheath by operation of a control knob. The stopper can be configured to abut a distal end of the inner sheath when the clip is in a closed state which prevents the wire from locking the clip and the inner sheath can be configured to move proximally to allow operating the wire to move proximally to lock the clip. In other embodiments, the clip device can further include a stopper configured to stop the wire from moving proximally. In some embodiments, the clip device can further include a handle having a main body and a slit with a slider located in the slit. The slider can be connected to a proximal end of the wire and the can be stopper located in the slit to prevent the slider from moving to a proximal end of the slit. In other embodiments, the stopper are protrusions that face each other on the first arm and second arm and the device further includes a tube with a pin in which the pin abuts the stoppers when the clip is retracted proximally in the tube.

Additional advantages of the present invention will become readily apparent to those skilled in this art from the following detailed description, wherein only the preferred embodiment of the invention is shown and described, simply by way of illustration of the best mode contemplated of carrying out the invention. As will be realized, the invention is capable of other and different embodiments, and its several details are capable of modifications in various obvious respects, all without departing from the invention. Accordingly, the drawings and description are to be regarded as illustrative in nature, and not as restrictive.

### BRIEF DESCRIPTION OF THE DRAWINGS

Reference is made to the attached drawings, wherein elements having the same reference numeral designations represent similar elements throughout and wherein:

FIG. 1 schematically illustrates a clip device with a clip having latch parts protruding from arms of the clip and configured to lock the clip in a closed state by pulling a wire in a single operation.

FIGS. 2A, 2B, 2C and 2D schematically illustrate a clip device with a latch element on a clip arm configured to lock the clip in a closed state by slidably moving a locking ring over the latch element with a control member in accordance with aspects of the present disclosure.

FIGS. 3A, 3B, and 3C schematically illustrate a clip device with latch elements on clip arms and a locking ring complementary mating portions to the latch elements which lock the ring to the latch elements and lock the clip in a closed state in accordance with aspects of the present disclosure.

FIG. 3D is an enlarged view of the locking ring with complementary mating portions to the latch elements on the clip arms of FIG. 3A.

FIGS. 4A, 4B, 4C, and 4D schematically illustrate a clip device including a clip having a first and second arm in which the first arm includes a first latch element and the second arm includes a second latch element opposite the first latch element and configured to engage the first latch element to lock the clip in accordance with aspects of the present disclosure.

FIGS. 5A, 5B, 5C, and 5D schematically illustrate a clip device including a clip, a wire connected to a proximal end of the clip, a stopper connected to a distal end region of the wire and an inner sheath reciprocally movable within a sheath by operation of a control knob in accordance with aspects of the present disclosure.

FIGS. 6A and 6B schematically illustrate a clip device including a removable stopper on an operational handle of the device in accordance with aspects of the present disclosure.

FIGS. 7A, 7B and 7C schematically illustrate another clip device including a stopper in accordance with aspects of the present disclosure.

FIGS. 8A and 8B schematically illustrate another clip device including a stopper in accordance with aspects of the present disclosure.

FIGS. 9A and 9B schematically illustrate another clip device including a stopper in accordance with aspects of the present disclosure.

FIGS. 10A and 10B schematically illustrate another clip device including a stopper in accordance with aspects of the present disclosure.

Throughout all of the drawings, dimensions of respective constituent elements are appropriately adjusted for clarity. For ease of viewing, in some instances only some of the named features in the figures are labeled with reference numeral.

### DETAILED DESCRIPTION OF THE DISCLOSURE

Clips of the present disclosure are useful in therapeutic procedures of a patient such as to prevent tissue bleeding, closing perforations and hemostasis, and suture contraction of an inner wound, marking lesions and traction (mucosal elevation) and other surgical procedures. Generally a clip device for grasping tissue of a patient includes a clip that can be actuated by a wire configured in a sheath in which the wire can advance and retract longitudinally within the sheath by operating a handle of the device. References herein to the term “distal” are to a direction away from the operating handle, while references to the term “proximal” are to a direction towards the operating handle. For example, US 2019/0104926, which is incorporated by reference herein, discloses an endoscope treatment tool having clips that can lock. The term “patient,” as used herein, comprises any and all organisms and includes the term “subject.” A patient can be a human or an animal.

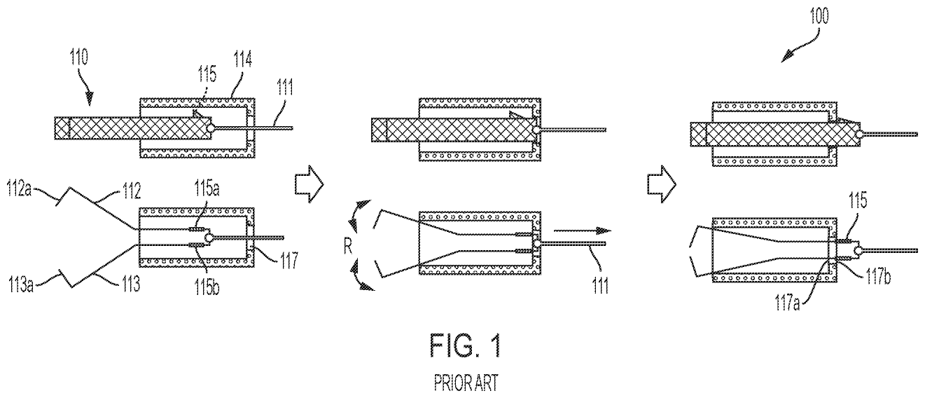

FIG. 1 schematically illustrates a clip device 100 configured to lock a clip by pulling a wire in a single operation. As shown, such a device includes a clip 110 having arms 112, 113, for grasping tissue and claws 112a, 113a at distal ends of the arms 112, 113, respectively. The device further includes a wire 111 connected to a proximal end of the clip 110 and configured to reciprocally move within a sheath (not shown) and a tube or pipe 114 connected to a distal end of a sheath (not shown)(the terms tube and pipe are used interchangeably herein). During operation, arms 112 and 113, which are biased toward an open, tissue receiving configuration, can be deployed from pipe 114 and retracted into pipe 114 by reciprocally moving wire 111. For example, as the wire 111 is pulled proximally, arms of the clip 110 are drawn into the pipe 114 so that contact with the pipe 114, such as contact with the edges forming the opening of the pipe 114, draws distal ends of the arms toward one another to close the clip arms and, as the wire is pushed distally, arms of the clip 110 are deployed from the pipe 114 so that distal ends of the arms move away from one another due to the natural bias of the arms 112, 113 to be in an open state. Hence, the arms 112, 113 of the clip 110 can be reciprocally (R) closed and opened by reciprocally moving the wire 111 connected to the clip 110 proximally or distally.

As further illustrated in FIG. 1, clip 110 includes a latch part 115 protruding on an outer surface of at least one arm and a corresponding latch part 117 at a proximal end of pipe 114 such that when wire 111 is pulled proximally, latch part 115 moves from a distal surface 117a to a proximal surface 117b of corresponding latch part 117 of pipe 114 to lock the clip 110 in a closed state. Such a configuration allows the wire 111 to close the clip arms 112, 113 and lock the clip 110 in a single operation by simply pulling the wire 111 proximally without interruption. However, users of such a clip device 100 can unintentionally lock the clip 110 when operating the wire 111 during a therapeutic procedure.

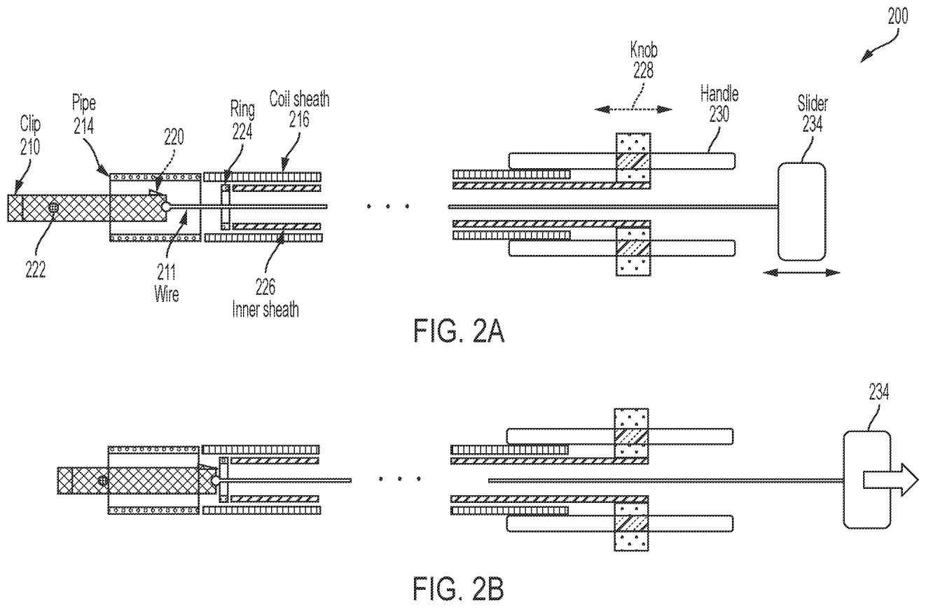

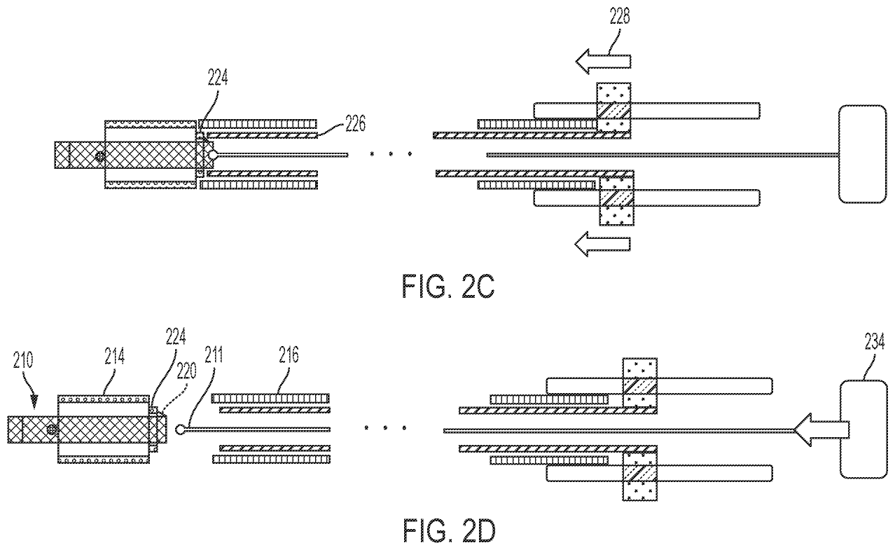

An advantage of the present disclosure includes a device, e.g., treatment tool, that includes a clip that can be reciprocally closed and opened in one operation and locked in a separate operation, distinct from the operation of reciprocally closing and opening the clip, thereby minimizing unintended locking of the clip while operating the device. For example, the clip can be locked by an operation other than operating the wire to close and open the clip such as by operating a stopper or by operating a control member reciprocally movable within the sheath to slid over the clip arms or to slid a locking ring over the clip arms. Various embodiments and aspects of a clip device according to the present disclosure will be described with references to FIGS. 2 through 10.

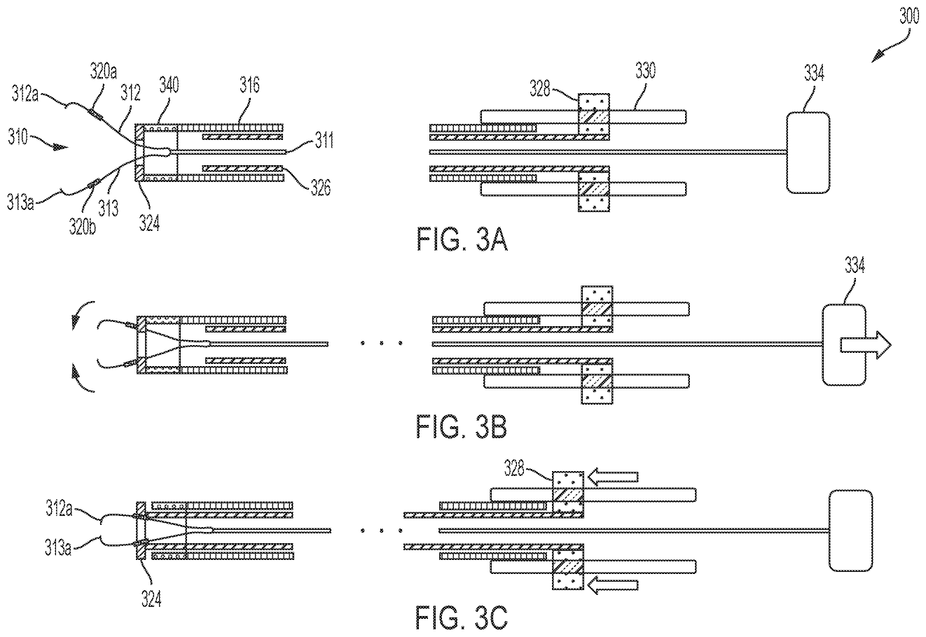

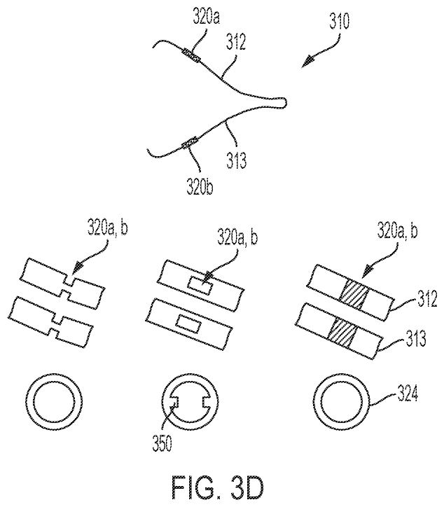

FIGS. 2-3 schematically illustrate a clip device with one or more latch elements on a clip arm configured to lock the clip in a closed state by slidably moving a locking ring over the one or more latch elements with a control member in accordance with aspects of the present disclosure. For example, and as shown in FIGS. 2A, 2B, 2C and 2D, a clip device 200 can include clip 210 and wire 211 connected to a proximal end of the clip 210 and configured to reciprocally move within coil sheath 216 to retract and deploy clip 210 from pipe 214 disposed at a distal end of sheath 216 to close and open the clip 210. The arms of such a clip 210 are biased toward an open, tissue receiving configuration. A proximal end of the wire 211 is connected to and operated by slider 234 housed on handle 230. Slider 234 can reciprocally move wire 211 within coil sheath 216 to retract and deploy clip 210 from pipe 214.

In this example, the device further includes a locking ring 224 housed in the coil sheath 216 and a control member, e.g., an inner sheath, 226 reciprocally movable within the coil sheath 216 by reciprocal operation of a control knob 228. The clip 210 further includes a latch element 220 protruding therefrom and also includes projection 222 at a distal end region of at least one arm of the clip. As shown in FIGS. 2A-2B, clip 210 can be drawn into pipe 214 to close the clip arms and also clip 210 can be deployed from pipe 214 to open the clip arms by operation of the wire 211 through slider 234 without locking the clip 210. For this example, wire 211 is configured to reciprocally move within the control member, e.g., inner sheath. Projection 222 limits the clip 210 from moving too far proximally in pipe 214 as shown in FIG. 2B. Thus a separate operation is needed to lock the clip 210.

As shown in FIGS. 2A-2D, locking ring 224 is configured to slidably move over latch element 220 and be disposed between pipe 214 and latch 220 in an axial direction to lock clip 210 in pipe 214. As shown in FIG. 2C, while the clip 210 is retracted within pipe 214 and is in a closed state, locking ring 224 can be pushed distally over latch element 220 by sliding control member 226 distally with control knob (228). Once locking ring 224 is pushed distally over latch element 220, arms of the clip 210 are locked in a closed state and cannot be readily opened. Once the clip is locked, the clip 210 and pipe 214 can be disconnected from the coil sheath 216 as shown in FIG. 2D. Certain clip devices can include a link mechanism that can engage and release a pipe from the sheath such as in a QuickClip pro (QCP) clip having a connection member (not shown). See, e.g., U.S. Pat. No. 9,949,740, which is incorporated herein by reference. Such devices include a connection member to engage and disengage a distal end of the clip 210 from the wire 211.

In another example, FIGS. 3A, 3B, and 3C schematically illustrate a clip device 300 with latch elements on clip arms and a locking ring having complementary mating portions to the latch elements which lock the ring to the latch elements and lock the clip in a closed state in accordance with aspects of the present disclosure. As shown in FIGS. 3A, 3B and 3C, the clip device includes clip 310 including a first arm 312, and a second arm 313, which are biased toward an open, tissue receiving configuration, and claws 312a, 313a, at distal ends of the arms 312, 313, respectively. In addition, the first arm 312 of the clip 310 has a first latch element 320a and the second arm 313 has a second latch element 320b. A wire 311 is connected to a proximal end of the clip 310 and configured to reciprocally move within coil sheath 316. A slider 334 housed on handle 330 is connected to a proximal end of the wire 311 to operate the wire 311.

In this example, tube 340 is disposed at a distal end of sheath 316. Tube 340 can be a pipe, which can be disconnectable from sheath 316, or fixed onto sheath 316 without being able to be disconnected from the sheath. In additional embodiments, tube 340 is integral with sheath 316 and constitutes a distal end region of sheath 316. Integrally formed tube 340 is stiffer than sheath 316 so that the distal end provides a sufficiently rigid bearing surface to interact with arms 312, 313 and cause them to move toward each other when the clip arms 312, 313 are drawn into the distal end region. For example, the distal end region does not expand when the clip arms 312, 313 are drawn into the distal end region. The device further includes a locking ring 324 located at a distal end of tube 340 and a control member, e.g., an inner sheath, 326 reciprocally movable within the coil sheath 316 by operation of a control knob 328. The locking ring 324 can be held in place by the clip arms 312, 313 which are biased in an open state and which can apply a constant force against the locking ring 324 toward the distal end of the sheath.

As shown in FIGS. 3A-3B, clip 310 can be drawn into tube 340 to close the clip arms 312, 313 and can also be deployed from the tube to open the clip arms 312, 313 by operation of the wire 311, which is configured to reciprocally move within the control member through slider 334 without locking the clip 310. In a separate operation, as shown in FIG. 3C, while the clip 310 is retracted within tube 340 and is in a closed state, locking ring 324 can be pushed distally over latch elements 320a and 320b by sliding control member 326 distally with control knob 328. Once locking ring 324 is pushed distally over latch elements 320a, 320b, arms 312, 313 of the clip 310 are locked in a closed state and cannot be readily opened. In addition, latch elements 320a, 320b can have complementary mating portions to locking ring 324 which lock the locking ring 324 to the one or more latch elements thereby locking the clip 310. FIG. 3D illustrates three different examples of latch elements 320 on clip arms 312, 313 of clip 310, and complementary mating portions 350 on locking rings 324.

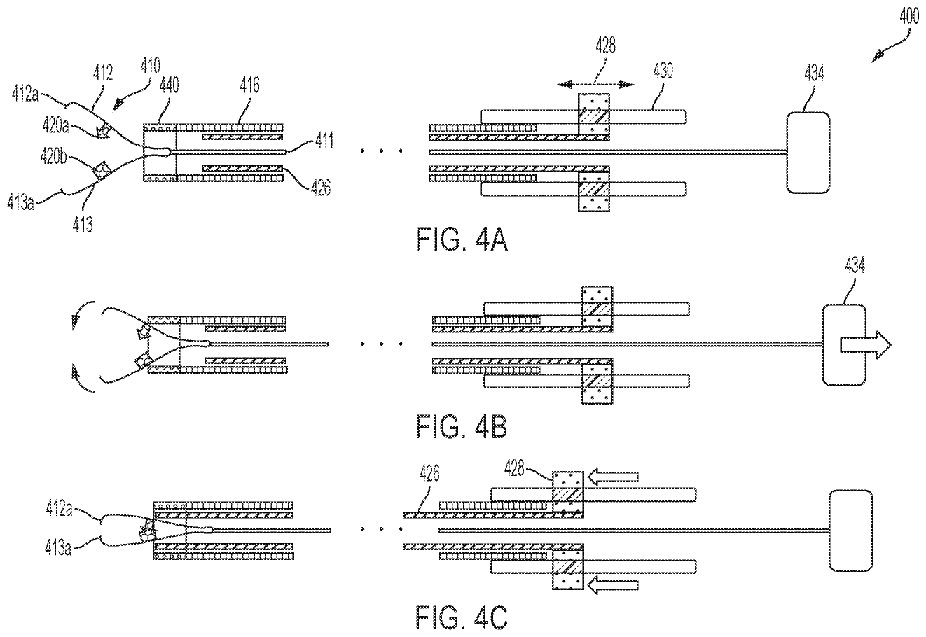

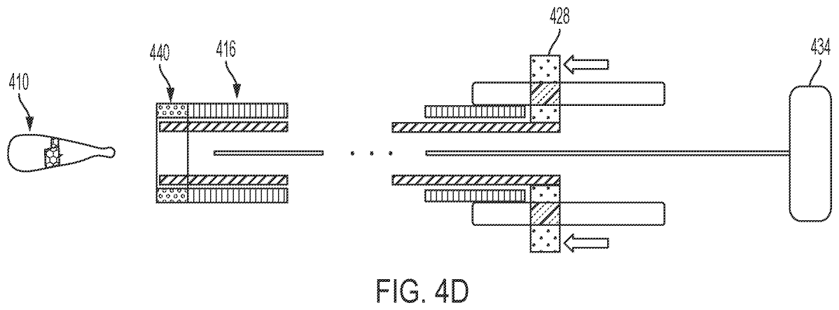

FIGS. 4A-4D schematically illustrate another example of a clip device 400 in which different structural elements are used to lock the clip and to close and open the clip. As shown in FIGS. 4A, 4B and 4C, clip device 400 includes clip 410 and a wire 411 connected to a proximal end of the clip 410 and configured to reciprocally move within coil sheath 416. Clip 410 includes a first arm 412, and a second arm 413, which are biased toward an open, tissue receiving configuration, and claws 412a, 413a, at distal ends of the arms 412, 413, respectively. The wire 411 is operated by slider 434 housed on handle 430 and the slider 434 is connected to a proximal end of the wire 411. Tube 440 is connected to a distal end of sheath 416. In this example, tube 440 is fixed onto sheath 416 without being configured to disconnect from the sheath. In alternative embodiments, tube 440 can be a separate element attached to sheath 416 or can be integrally formed with sheath 416, i.e., as a distal end region of the sheath.

As shown in FIGS. 4A and 4B, clip 410 can be drawn into tube 440 to close the clip arms 412, 413 and also can be deployed from the tube to open the clip arms 412, 413 by operation of the wire 411 through slider 434 without locking the clip 410. In this example, clip 410 includes a first arm 412 which has a first latch element 420a and a second arm 413 which has a second latch element 420b. The first and second latch elements 420a, 420b, oppose each other and are configured to engage each other and lock when pressed together. As shown in FIG. 4C, while the clip 410 is retracted within tube 440 and is in a closed state, control member, e.g., inner sheath, 426 can be slid distally with control knob 428 over clip arms 412, 413, to press and lock the first and second latch elements 420a, 420b together. Once inner sheath 426 is pushed distally over latch elements 420a, 420b, the latch elements engage to lock the arms 412, 413 of the clip 410. Once locked, the arms 412, 413 cannot be readily opened. Once the clip 410 is locked, the clip 410 itself can be disconnected from the wire 411 as shown in FIG. 4D. In this example, no pipe is released when the clip 410 is released from the sheath 416.

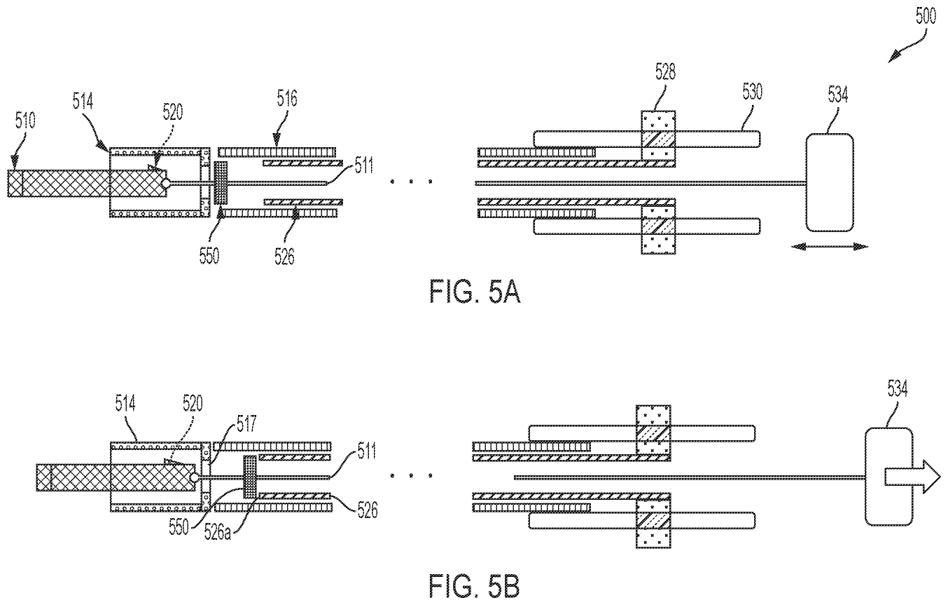

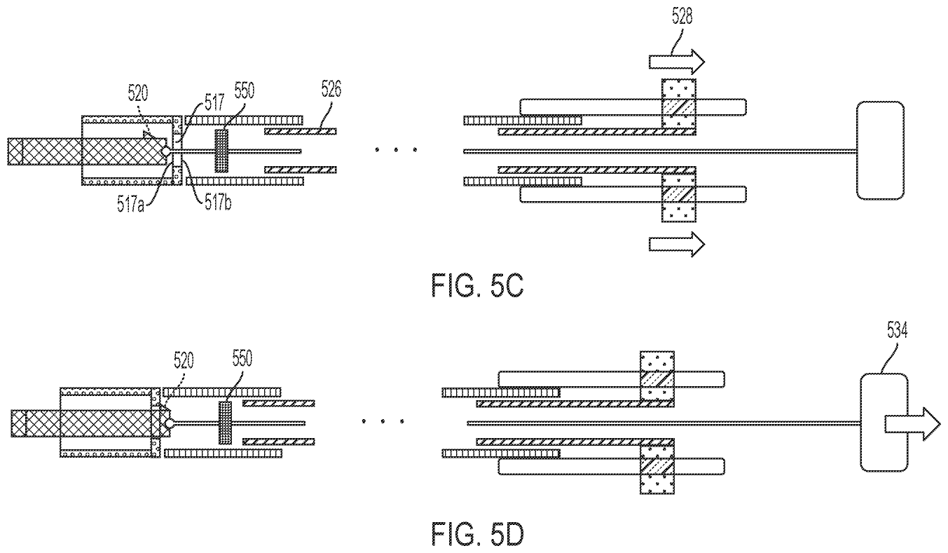

FIGS. 5A-5D schematically illustrate another example of a clip device in which operating a wire does not lock the clip in a closed state in a single operation. As shown in FIGS. 5A, 5B, 5C, and 5D, a clip device 500 includes clip 510, a wire 511 connected to a proximal end of the clip 510 and configured to reciprocally move within a sheath 516, and a pipe 514 connected to a distal end of a sheath 516. The wire 511 is operated by slider 534 housed on handle 530 and the slider 534 is connected to a proximal end of the wire 511. The clip device 500 further includes a control member, e.g., an inner sheath, 526 reciprocally movable within the sheath 516 by operation of a control knob 528. A stopper 550 is connected to a distal end region of wire 511.

As further illustrated in FIG. 5A-5B, clip 510 includes a latch part 520 protruding on an outer surface of at least one arm of the clip 510 and a corresponding latch part 517 at a proximal end of pipe 514. However, the wire 511, stopper 550, and inner sheath 526 are configured to prevent locking of the clip, e.g., by pulling latch part 520 past corresponding latch part 517 such that the protruding latch part 520 engages with corresponding latch part 517 to prevent movement of the clip 510 in a distal direction, in a single operation by simply pulling the wire proximally without interruption.

As shown in FIG. 5B, the stopper 550 is configured to abut a distal end 526a of the inner sheath 526 when the clip 510 is in a closed state and when the inner sheath 526 is positioned distally. Such a configuration prevents the wire 511 from locking the clip 510 when the wire 511 and slider 534 are pulled proximally to close clip 510 in pipe 514. Such a configuration prevents a user from unintentionally locking the clip 510 when closing the clip during operating the device.

As shown in FIGS. 5C-5D, the inner sheath 526 is configured to move proximally by operating knob 528. When positioned proximally, the inner sheath 526 allows stopper 550 to move proximally when further moving wire 511 and slider 534 proximally. After moving inner sheath 526 proximally, a user can move wire 511 proximally such that when wire 511 is pulled proximally latch part 520 moves from a distal surface 517a to a proximal surface 517b of corresponding latch part 517 of pipe 514 to lock the clip 511 in a closed state.

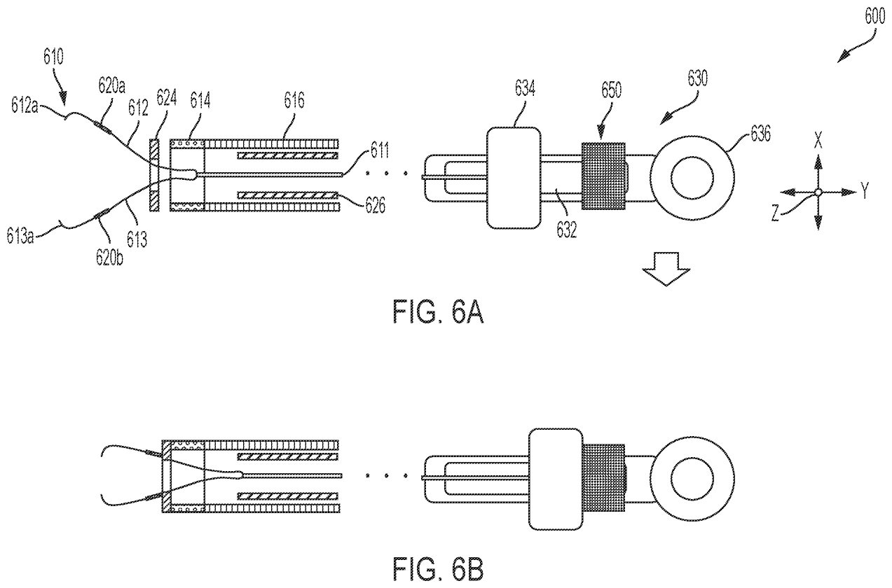

FIGS. 6-10 illustrate other stopper configurations with clip devices according to aspects of the present disclosure. Such stopper configuration can be included with one or more features of devices illustrated in FIGS. 2-5.

For example, FIGS. 6-9 illustrate a clip 610, including a first arm 612, having a first latch element 620a, second arm 613 having a second latch element 620b, and wire 611 connected to a proximal end of the clip and configured to reciprocally move within coil sheath 616. The clip arms 612, 613, further include claws, 612a, 613a, at distal ends of the clip arms 612, 613. During operation, clip arms 612 and 613, which are biased toward an open, tissue receiving configuration, can be deployed from and retracted into a tube or pipe by reciprocally moving wire 611. The device further includes a control member, e.g., an inner sheath, (626 or 826) and locking ring 624 which can be slid over the latch elements 620a, 620b of the clip arms 612, 613 by operation of the inner sheath. A slider 634 is connected to a proximal end of wire 611 to operate the wire 611.

As further shown in the example of FIGS. 6A-6B, the clip device 600 includes a handle 630 connected to a proximal end portion of a sheath 616. The handle 630 in this example is in the form of a bar shape extending in the axial direction Y having a finger hooking part 636 at the proximal end region thereof and a slit 632 extending in the axial direction Y.

In this device, a removable stopper 650 is included in the slit 632 at a proximal end of the slit to prevent the slider 634 from reaching the proximal end of the slit 632 when the stopper 650 is in place. In such a configuration, the clip 610 cannot be inadvertently locked by pulling the slider 634 to close the clip 610. Additional operations are needed to lock the clip 610 in a closed state for the configuration of this example, which include removing the stopper 650 and moving the inner sheath 626 to push locking ring 624 distally over latch elements 620a and 620b. Once lock ring 624 is pushed distally over latch elements 620a, 620b, arms 612, 613 of the clip 610 are locked in a closed state and cannot be readily opened.

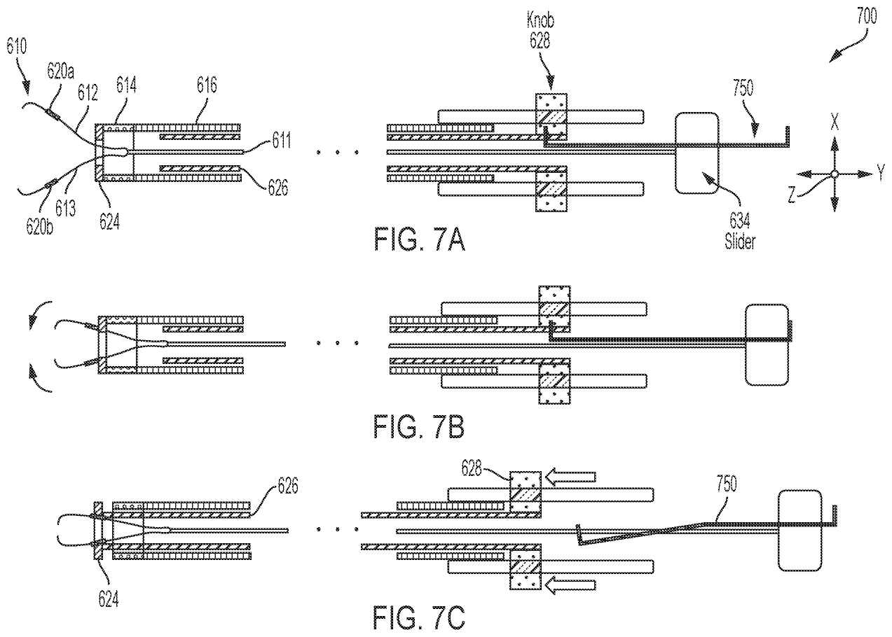

FIGS. 7A, 7B, and 7C schematically illustrate another clip device 700 including a stopper structure in accordance with aspects of the present disclosure. As shown, stopper 750 is a restraining device that is reversibly connected to knob 628 and extends to a proximal length of where the slider 634 moves proximally to close the clip 610. When stopper 750 is connected to knob 628, the stopper 750 prevents inner sheath 626 from moving distally to lock the clip 610 when the clip in in a closed state as shown in FIG. 7B. To lock the clip, stopper 750 is released from the knob 628 and the knob 628 and inner sheath 626 are moved distally to push locking ring 624 over latch elements 620a and 620b.

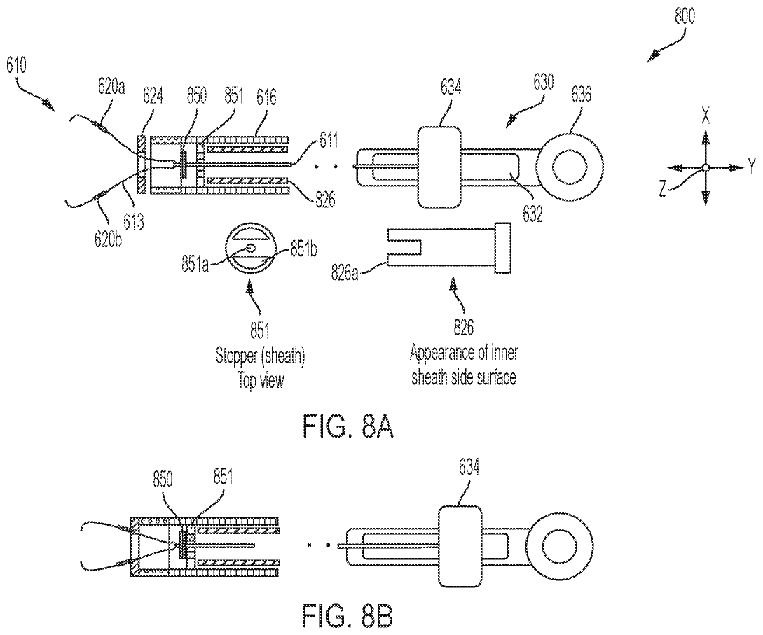

FIGS. 8A-8B schematically illustrate another clip device 800 including a stopper and inner sheath structure in accordance with aspects of the present disclosure. As shown in this example, stopper 850 is connected to a distal end region of wire 611. An additional stopper 851 is housed in and connected to sheath 616 at a location proximal to the stopper 850 on wire 611. The stopper 851 connected to the sheath 616 can have a port 851a, typically in a center of a cross-section, to allow the wire 611 to reciprocally move through the stopper 851 and ports 851b, typically off-center, to allow the inner sheath to reciprocally translate through the stopper 851. The inner sheath 826 is configured to have extending portions 826a to translate past stopper 851 and push locking ring 624 over latch elements 620a and 620b when locking the clip 610. As further shown in FIG. 8B, when wire 611 is moved proximally by slider 624 to close the clip arms 612, 613, stopper 850 abuts the additional stopper 851 in the sheath 616 preventing the clip 610 from inadvertently being locked. To lock the clip 610, inner sheath 826 is pushed through additional stopper 851, e.g., though one or more ports 851b, to push locking ring 624 over latch elements 620a and 620b.

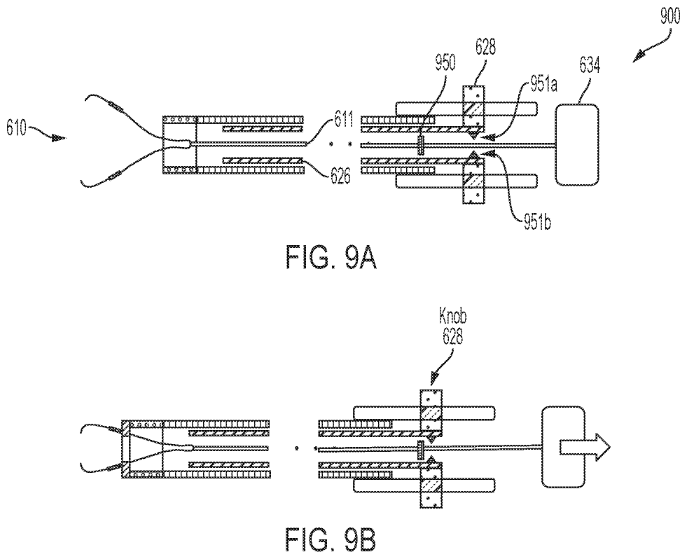

FIGS. 9A and 9B schematically illustrate another clip device 900 including stopper structures in accordance with aspects of the present disclosure. As illustrated in this example, the device includes a stopper 950 connected to wire 611 located at a proximal region of the wire 611 and stoppers 951a, 951b on inner sheath 626. Stoppers 951a, 951b project inward relative to the inner surface of the inner sheath 626 For this example, the stoppers 951a, 951b on inner sheath 626 are located at knob 628. Further, inner stoppers 951a, 951b are configured in a spaced-apart relation to allow the wire 611 to translate through a space between the inner stoppers 951a, 951b. However, the size of the space is less than a size of the stopper 950 such that, when translated proximally by wire 611, the stopper 950 abuts inner stoppers 951a, 951b and is stopped from further moving in the proximal direction. However, the wire stopper 950 can translate through the space between the inner stoppers 951a, 951b with sufficient force applied to the wire 611, which may assist in assembly of the wire in the clip device. In some embodiments, at least a portion of one or both of the stopper 950 and the inner stoppers 951a, 951b can be made of resilient material, such as rubber.

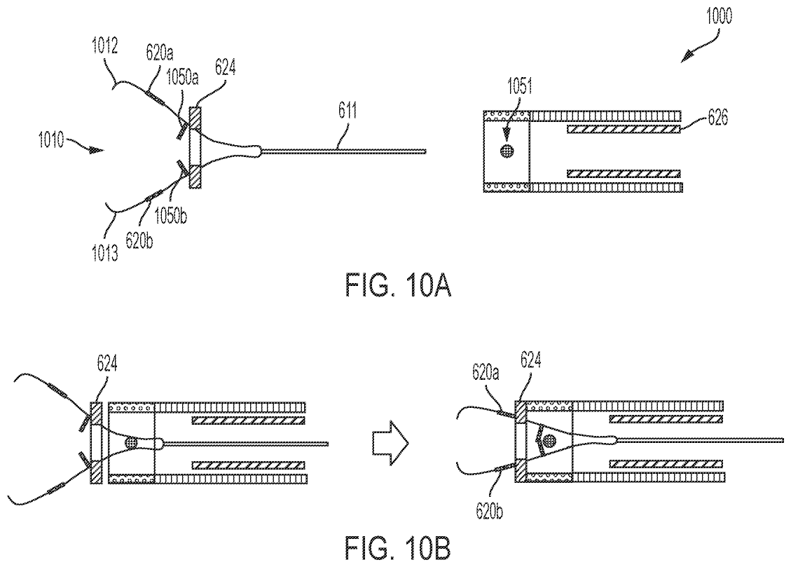

FIGS. 10A and 10B schematically illustrate another clip device 1000 including a stopper in accordance with aspects of the present disclosure. As shown in this example, clip 1010 includes two opposing protrusions facing each other. The clip 1010 includes a first arm 1012 with a first inner protrusion 1050a, a second arm 1013 with a second inner protrusion 1050b, in which the protrusions are on inner surfaces of the arms 1012, 1013 and face each other. The tube or pipe in this example further includes a pin 1051 configured to abut the protrusions 1050a, 1050b on the clip arms 1012, 1013 when the clip 1010 is retracted in the tube or pipe. The interaction of the protrusions 1050a, 1050b with the pin 1051 limits the distance that the clip 1010 can retract into the tube or pipe. In such a configuration, the clip 1010 cannot be inadvertently locked by pulling the wire 611 to close the clip 1010. Rather, to lock the clip 1010, the inner sheath 626 is pushed in the distal direction to move locking ring 624 distally over latch elements 620a and 620b

As illustrated by the various examples, a clip device can be operated by closing a clip having a first arm and second arm biased in an open state and having one or more latch elements by pulling a wire connected to a proximal end of the clip to retract the clip in a tube or pipe in one operation. In a second operation, the clip can be locked in a closed state by moving a stopper or by an operation other than pulling the wire to close the clip. For example, locking the clip can be carried out by sliding a locking ring distally over the one or more latch elements or by sliding an inner sheath over the clip arms to lock mating latch elements on the clip arms.

While the claimed invention has been described in detail and with reference to specific embodiments thereof, it will be apparent to one of ordinary skill in the art that various changes and modifications can be made to the claimed invention without departing from the spirit and scope thereof. Thus, for example, those skilled in the art will recognize, or be able to ascertain, using no more than routine experimentation, numerous equivalents to the specific substances and procedures described herein. Such equivalents are considered to be within the scope of this invention, and are covered by the following claims.

What is claimed is:

1. A clip device comprising:
a clip having a first arm and second arm in which the first arm and second arm are configured to move in a direction toward and away from each other to close and open the clip;
a wire connected to a proximal end of the clip configured to reciprocally move within a sheath to close and open the clip;
one or more latch elements on the clip configured to lock the clip in a closed state;
wherein operating the wire cannot lock the clip in a closed state in a single operation.

2. The clip device of claim 1, further comprising a locking ring that can slidably move over the one or more latch elements to lock the clip.

3. The clip device of claim 2, wherein the first arm or second arm or both include a projection that prevents the arms from fully retracting in a tube disposed at a distal end of the sheath.

4. The clip device of claim 2, wherein the locking ring and the one or more latch elements have complementary mating portions which lock the locking ring to the one or more latch elements and to lock the clip.

5. The clip device of claim 2, further comprising a control member reciprocally movable within the sheath by operation of a control knob that is configured to slidably move the locking ring over the one or more latch elements to lock the clip.

6. The clip device of claim 5, wherein the control member is an inner sheath and the wire is configured to reciprocally move within the inner sheath to close and open the clip.

7. The clip device of claim 2, further comprising a tube disposed at a distal end of the sheath, wherein the wire connected to the proximal end of the clip is configured to reciprocally move the clip within the tube to close and open the clip and wherein the locking ring is configured to slidably move over the one or more latch elements and be disposed between the tube and the one or more latch elements in an axial direction to lock the clip.

8. The clip device of claim 1, wherein the first arm includes a first latch element and the second arm includes a second latch element opposite the first latch element and configured to engage the first latch element to lock the clip.

9. The clip device of claim 8, further comprising an inner sheath reciprocally movable within the sheath by operation of a control knob that can slidably move over the first arm and second arm to engage the first and second latch elements to lock the clip.

10. The clip device of claim 8, further comprising a tube connected to a distal end of the sheath, which does not disconnect from the sheath.

11. The clip device of claim 1, further comprising a stopper connected to a distal end region of the wire and an inner sheath reciprocally movable within the sheath by operation of a control knob, wherein the stopper is configured to abut a distal end of the inner sheath when the clip is in a closed state which prevents the wire from locking the clip and the inner sheath is configured to move proximally to allow operating the wire to move proximally to lock the clip.

12. The clip device of claim 1, further comprising a stopper configured to stop the wire from moving proximally.

13. The clip device of claim 11, further comprising a handle having a main body and a slit with a slider located in the slit, wherein the slider is connected to a proximal end of the wire and the stopper is located in the slit to prevent the slider from moving to a proximal end of the slit.

14. The clip device of claim 12, wherein the stopper is connected to the wire.

15. The clip device of claim 12, wherein the stopper are protrusions that face each other on the first arm and second arm and the device further includes a tube with a pin in which the pin abuts the stoppers when the clip is retracted proximally in the tube.

16. A method of operating a clip device, the method comprising
closing a clip having a first arm and second arm biased in an open state and having one or more latch elements by pulling a wire connected to a proximal end of the clip to retract the clip in a tube; and
locking the clip in a closed state by moving a stopper or by an operation other than pulling the wire to close the clip.

17. The method of claim 16, further comprising locking the clip by sliding a locking ring distally over the one or more latch elements.

18. The method of claim 16, further comprising locking the clip by sliding an inner sheath over the clip to lock mating latch elements.

19. A clip device comprising:
a clip having a first arm and second arm in which the first arm and second arm are configured to move in a direction toward and away from each other to close and open the clip;
a wire connected to a proximal end of the clip configured to reciprocally move within a sheath to close and open the clip; and
one or more latch elements on the clip configured to lock the clip in a closed state;
wherein a first operation of the clip device reciprocally moves the wire within the sheath to close and open the clip,
wherein a second operation of the clip device locks the clip in a closed state, and wherein the first operation is distinct from the second operation.

20. The clip device of claim 19, wherein the second operation occurs without operating the wire.
